(19) United States
(12) Patent Application Publication
(10) Pub. No.: US 2024/0255338 A1
(43) Pub. Date: Aug. 1, 2024

(54) SCENT SOLUTION WEIGHT LOSS TESTING

(71) Applicant: Pura Scents, Inc., Pleasant Grove, UT (US)

(72) Inventors: Trevor Davis, American Fork, UT (US); Rachel Beckstead, Lehi, UT (US); David Freitag, Pleasant Grove, UT (US); Hannah Trimble, Salt Lake City, UT (US); Juan Carlos Altuna, South Jordan, UT (US); Celeste Chubak, West Valley City, UT (US); Jeremy Hubbard, Paradise, UT (US)

(21) Appl. No.: 18/425,597

(22) Filed: Jan. 29, 2024

Related U.S. Application Data
(60) Provisional application No. 63/481,947, filed on Jan. 27, 2023.

(51) Int. Cl. G01F 23/20

(52) U.S. Cl. CPC G01F 23/20

(57) ABSTRACT

A scent solution weight loss device including a housing configured to retain a scent solution, a sensor configured to detect weight loss data of the scent solution, a heating element configured to heat the scent solution, and an output device configured to provide the weight loss data to a weight loss application, wherein the weight loss application is configured to determine a scent solution lifetime based on the weight loss data.

### BACKGROUND

The present disclosure relates to determining a lifetime of a scent solution in a scent dispenser unit.

Existing solutions for determining how much scent is remaining in a scent liquid require elaborate setups where multiple scent dispensers are setup for manual testing. At regular intervals, the remaining scent liquids in the multiple scent dispensers are manually measured to track the scent liquid remaining in each of the scent dispensers. These manual measurements are then used to estimate the average lifetimes of the scent liquid. These existing solutions suffer from requiring intensive manual measurements at various intervals in order to capture the scent remaining. These manual measurements suffer from inconsistencies and human errors during measurement, as well as also being labor intensive.

### SUMMARY

In some aspects, the techniques described herein relate to a scent solution weight loss device, including a housing configured to retain a scent solution; a sensor configured to detect weight loss data of the scent solution as the scent solution is emitted; and an output device configured to provide the weight loss data to a weight loss application, wherein the weight loss application is configured to determine a scent solution lifetime based on the weight loss data.

In some aspects, the techniques described herein relate to a scent solution weight loss device, wherein the weight loss application is configured to generate a scent solution profile using the weight loss data.

In some aspects, the techniques described herein relate to a scent solution weight loss device, wherein the scent solution profile shows a change in scent solution over a measured time.

In some aspects, the techniques described herein relate to a scent solution weight loss device, wherein the sensor is further configured to measure a temperature of the scent solution.

In some aspects, the techniques described herein relate to a scent solution weight loss device, wherein the scent solution profile further includes the measured temperature of the scent solution.

In some aspects, the techniques described herein relate to a scent solution weight loss device, wherein the housing further includes a heating element configured to heat the scent solutions to a temperature.

In some aspects, the techniques described herein relate to a scent solution weight loss device, wherein the weight loss data includes a detected temperature, a detected intensity, and a detected duration.

In some aspects, the techniques described herein relate to a scent solution weight loss device, wherein the weight loss application determines the scent solution lifetime based on the detected temperature, the detected intensity, and the detected duration.

In some aspects, the techniques described herein relate to a method of determining a scent solution lifetime, the method including receiving from a first weight loss testing device, first weight loss data for a first scent solution in a first container, the first weight loss data including a change in weight of the first scent solution in the first container, a first weight loss device temperature setting, a first weight loss device intensity setting, and a first weight loss device duration; receiving from a second weight loss testing device, second weight loss data for a second scent solution in a second container, the second weight loss data including a change in weight of the second scent solution in the second container, a second weight loss device temperature setting, a second weight loss device intensity setting, and a second weight loss device duration; and determining a first scent solution lifetime for the first scent solution using the first weight loss data and a second scent solution lifetime for the second scent solution using the second weight loss data.

In some aspects, the techniques described herein relate to a method, wherein the first scent solution and the second scent solution are a same scent type.

In some aspects, the techniques described herein relate to a method, further including: determining an overall scent solution lifetime based on the first scent solution lifetime and the second scent solution lifetime; and providing the overall scent solution lifetime to a scent application for display.

In some aspects, the techniques described herein relate to a method, wherein the first weight loss data is a first graph showing the change in the first scent solution in the first container over the first weight loss device duration and the second weight loss data is a second graph showing the change in the second scent solution in the second container over the second weight loss device duration.

In some aspects, the techniques described herein relate to a method, further including: determining an optimal scent solution lifetime based on the overall scent solution lifetime.

In some aspects, the techniques described herein relate to a method, wherein the first weight loss device temperature setting is different from the second weight loss device temperature setting.

In some aspects, the techniques described herein relate to a method, wherein the first weight loss device intensity setting is different from the second weight loss device intensity setting.

In some aspects, the techniques described herein relate to a method, wherein the first weight loss device duration is different from the second weight loss device duration.

In some aspects, the techniques described herein relate to a scent solution weight loss system, including: a weight loss device configured to receive a scent solution and measure weight loss data of the scent solution over time; a weight loss application configured to receive the weight loss data and generate a scent solution profile based on the weight loss data; and a scent dispenser configured to receive the scent solution profile and adjust one or more scent dispenser settings based on the scent solution profile.

In some aspects, the techniques described herein relate to a scent solution weight loss system, wherein scent dispenser settings include a temperature setting and an intensity setting and the scent dispenser adjusts one or more of the temperature setting and the intensity setting based on the scent solution profile.

In some aspects, the techniques described herein relate to a scent solution weight loss system, wherein the weight loss application determines a scent solution lifetime based on the weight loss data.

In some aspects, the techniques described herein relate to a scent solution weight loss system, wherein the scent dispenser adjusts one or more of the temperature setting and the intensity setting based on the scent solution lifetime to extend an operational time of the scent solution.

The features and advantages described herein are not all-inclusive and many additional features and advantages will be apparent to one of ordinary skill in the art in view of the figure and description. Moreover, it should be noted that the language used in the specification has been selected for readability and instructional purposes and not to limit the scope of the inventive subject matter.

### BRIEF DESCRIPTION OF THE DRAWINGS

The disclosure is illustrated by way of example, and not by way of limitation in the figures of the accompanying drawings in which like reference numerals are used to refer to similar elements.

FIG. 1 is a block diagram of an example system for scent dispenser weight loss testing.

FIG. 2 is a block diagram of an example scent dispenser.

FIGS. 3A-3B illustrate an example weight loss device.

FIGS. 4A-4B are various views of another example of a weight loss device.

FIG. 5 illustrates an example component of a weight loss device.

FIG. 6 illustrates an example system for weight loss testing.

FIG. 7 illustrates an example system for weight loss testing.

FIG. 8 illustrates an example method for weight loss testing.

FIG. 9 illustrates example GUIs for scent dispensation.

FIGS. 10A-10B illustrate another example system for weight loss testing.

### DETAILED DESCRIPTION

The technology described in this disclosure relates to using a weight loss device to determine scent lifetimes of various scent solutions being used to dispense scents from scent dispensers. As an example, the technology allows one or more weight loss devices to be attached to scent solutions, such as a scent vial with a liquid or a scent cartridge, and the one or more weight loss devices can measure and/or track the remaining scent solution in the scent dispenser over various scent dispensation levels, as well as at different temperatures, humidity levels, intensity levels, or other scent dispensation settings. In some implementations, the one or more weight loss devices can measure and track the remaining scent solution automatically and in substantially real-time with sampling rates in the seconds as compared to the previous solutions that required manual measurements at much longer intervals, such as once a day. In some implementations, the weight loss device can further provide the measurements and tracking data of the remaining scent solution to a weight loss application that can determine various scent lifetimes and trends and analytics based on the collected data. These scent lifetimes can be used to inform consumers of recommended dispensing levels and/or timelines to replace scent solutions and/or to inform producers how to maximize scent lifetimes at different dispensing levels and/or for different solutions.

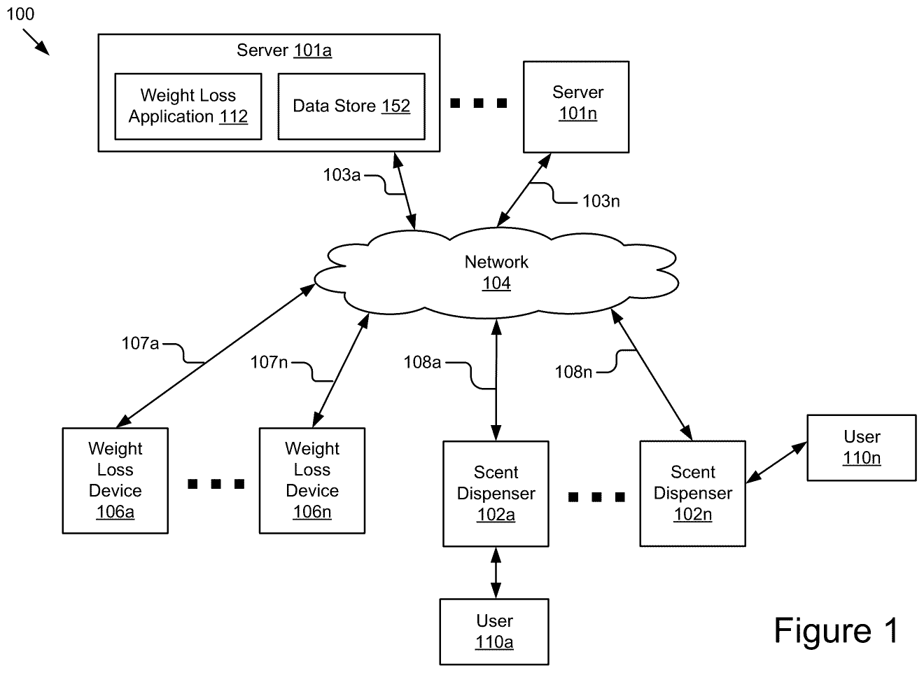

FIG. 1 is a block diagram illustrating an example system 100 for scent dispenser weight loss testing. The system 100 may include one or more weight loss devices 106 configured to measure and/or track scent solutions and the loss of the scent solutions over time at various settings. These weight loss devices 106 may be configured to receive one or more scent solutions containers, such as vials, and measure the remaining scent solutions as a scent is dispensed. The weight loss devices 106 may provide the measurements and/or tracking data to a weight loss application 112 connected to a server 101. As shown, the illustrated system 100 further includes scent dispensers 102 and a server 101, which are electronically communicatively coupled via the network 104 for interaction with one another, the weight loss devices 106, and/or the scent dispenser(s) 132, etc., using standard networking protocols, as reflected by signal lines 103, 107, and/or 108.

The weight loss application 112 operable by the server 101 can receive weight loss data from the one or more weight loss device(s) 106. The weight loss application 112 can analyze the various weight loss data and identify trends for the different scent solutions that are being measured and/or tested in the one or more weight loss device(s) 106. The weight loss application 112 can use these trends to determine lifetimes of the various scent solutions and provide various analytics to users and/or producers of the scent dispensers.

The server 101 includes a data store 152 storing various types of data used by the weight loss application 112. Example data types include weight loss data, device data, user data, etc. The weight loss data may include scent solution weight loss measurements, scent solution identities, weight loss device settings, etc. The device data may include a device model, a scent solution type, usage statistics, scent diffusion time, temperature variations, humidity settings, airflow changes, intensity levels, etc. The user data may include entries for the users 110 of the system 100. In some implementations, the user 110 may be a producer or manufacturer that is testing the weight loss device(s) 106 in volume, while in other implementations, the user 110 may be private consumer with a single or few weight loss device(s) 106. A given entry in the private consumer situation may include a unique identifier for the user, a unique identifier for a user device, contact information for the user (e.g., address, phone number, electronic address (e.g., email)), payment information, scent subscription information specifying which reoccurring scent cartridges should be shipped to the user, etc.

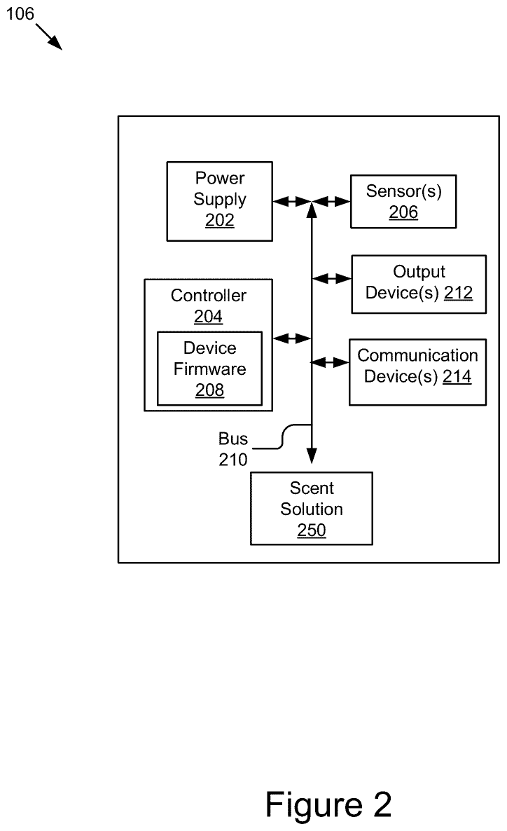

FIG. 2 is a block diagram illustrating an example weight loss device 106, which is depicted as including a power supply (PS) 202, one or more sensor(s) 206, a controller 204, output device(s) 212, device firmware 208, communication device(s) 214, and any number of scent solutions 250. The components 202, 204, 206, 212, 214, and 250 are communicatively coupled via a communications bus 210. The controller 204 may include a non-transitory memory device, or may be coupled to a non-transitory memory device also coupled for communication via the bus 210. The non-transitory memory device may store software that specially configures the controller, such as the device firmware 208. The PS 202 may be any AC and/or DC power supply for powering the weight loss device 106. In some implementations, the PS 202 may be battery powered and may be configured to charge when plugged into an AC and/or CD power supply. The controller 204 may be a microchip that controls the constituent electronics (e.g., sensor(s) 206, output device(s) 212, communication device(s) 214, etc.) of the weight loss device 106.

The one or more sensor(s) 206 may include one or more pressure sensors for detecting changes in weight of the weight loss device 106 as a scent solution 250 is used to dispense a scent over time. The pressure sensors may establish an initial baseline weight when a full scent solution 250, such as a scent liquid or gel stored in a vial or container, is positioned within the weight loss device 106 and then the one or more pressure sensors may measure changes in the weight of the scent solution 250 in substantially real-time as a scent is dispersed based on various settings of the weight loss device 106. In some implementations, the scent solution 250 may be heated using a heating component (not shown) at various dispensing settings to cause the scent solution 250 to evaporate and disperse as a scent. In further implementations, the scent solution 250 may be dispersed using a fan (not shown) at various fan speed levels to cause the scent solution (such as a liquid or gel) 250 to be dispersed as a scent. The one or more pressure sensors can detect the changes in the weight of the scent solution 250 to determine the remaining scent solution 250 and how the weight changes over time and/or dispensing settings.

The one or more sensor(s) 206 may include one or more temperature sensors for detecting the temperature of the scent solution 250 and/or ambient temperature adjacent to the weight loss device 106. The temperature sensors can detect these various temperatures and the weight loss device 106 can use those readings to determine efficient diffusion of the scent at different ambient temperatures and/or scent solution 250 temperatures. In other implementations, the one or more sensor(s) 206 may include humidity sensors to detect changes in humidity over time as the scent is dispensed. Other sensors 206 are also contemplated to provide environmental information, such as light readings, airflow readings, etc. that may effect the changes to scent during weight loss testing.

The communication device(s) 214 may include a transmitter or transceiver having a wireless interface configured to communicate with the devices coupled to the network 104, such as the server 101, and/or other components of the network 104 using standard communication protocols, such as Internet protocols. Further, the transceiver may be configured to wirelessly transmit data via a network to connect to other devices, such as a mobile device. By way of further example, the transceiver may transmit data to the mobile device to which it is linked using a protocol compliant with IEEE 802.15, such as Zigbee®, Z-Wave®, Bluetooth®, or another suitable standard. Further embodiments are also possible and contemplated. In some embodiments, the transceiver may be embedded in the controller 204 or may be a component distinct from the controller and coupled to the controller 204 via the bus 210. The transceiver may be able to provide automatic data in substantially real-time for an individual weight loss device 106 so that individual weight loss measurements for a scent solution 250 can be tracked in substantially real-time. In some implementations, as described elsewhere herein, where an array of weight loss devices 106 are used, each of the transceivers of the weight loss devices can provide data automatically to the weight loss application 112 related to each of the scent solutions 250 being tested and/or measured in each weight loss device 106 in the array of weight loss devices 106.

The output device(s) 212 may further include light sources and/or audio reproduction devices, although further suitable output devices are also contemplated and applicable. In some implementations, the light sources and/or audio reproduction devices may be controlled to produce output consistent with a scent being emitted by the scent dispenser (e.g., a low, soothing light and music may be output in conjunction with a relaxing scent being emitted), or to communicate various alerts, such as low power, low scent cartridge levels, etc. In further implementations, the output device(s) 212 may indicate various statuses of the weight loss device 106 during testing, such as a scent being emitted and measured, an indication of a temperature or intensity, an indication of a duration of the scent emission, an indication of a low level of scent remaining, an indication that the scent solution is empty, etc. These output device(s) 212 may be able to quickly signal to a user 110 the current status of a weight loss device 106, such as when using a large array of weight loss device(s) 106 in conjunction to test various weight loss data.

The scent solution 250 may be contained in a container, such as a vial or bottle for scent dispensing to diffuse a scent into the nearby air. In some implementations, the scent solution 250 may be a scent liquid that can be heated up to evaporate and diffuse a scent. In further implementations, the scent solution 250 may be a scent cartridge with an oil or gel that may diffuse a scent. In some implementations, the scent solution 250 may be used with a fan and/or heating elements to disperse the scent and test heating and airflow changes with different settings. The scent solutions 250 may have different scent profiles and the different scent profiles may have different weight loss data as the scent solution 250 is evaporated. The scent solution 250 may have various scent profiles and information about the scent solution 250 may be stored in the weight loss application 112 to store the various scent profiles, ages of the scent solutions 250, duration of use of the scent solutions 250, exposed temperatures of the scent solutions 250, etc.

Returning to FIG. 1, the server 101 may include one or more computing devices having data processing, storing, and communication capabilities. For example, the server 101 may include one or more hardware servers, virtual servers, server arrays, storage devices and/or systems, etc., and/or may be centralized or distributed/cloud-based. In some embodiments, the server 101 may include one or more virtual servers, which operate in a host server environment and access the physical hardware of the host server including, for example, a processor, memory, storage, network interfaces, etc., via an abstraction layer (e.g., a virtual machine manager).

While not depicted, the server 101 may include a (physical, virtual, etc.) processor, a non-transitory memory, a network interface, and a data store 152, which may be communicatively coupled by a communications bus. Similarly, a client device (not shown), such as a mobile device or computing device, may include a physical processor, a non-transitory memory, a network interface, a display, an input device, a sensor, and a capture device. It should be understood that the server and the client device may take other forms and include additional or fewer components without departing from the scope of the present disclosure.

Software operating on the server 101 (e.g., the weight loss application 160, an operating system, device drivers, etc.) may cooperate and communicate via a software communication mechanism implemented in association with a server bus. The software communication mechanism can include and/or facilitate, for example, inter-process communication, local function or procedure calls, remote procedure calls, an object broker (e.g., CORBA), direct socket communication (e.g., TCP/IP sockets) among software modules, UDP broadcasts and receipts, HTTP connections, etc. Further, any or all of the communication could be secure (e.g., SSH, HTTPS, etc.).

As shown, the server 101 may include a weight loss application 112 embodying a remotely accessible scent service. The weight loss application 112 may send data to and receive data from the other entities of the system including the weight loss device 106 and/or the scent dispenser 102, etc. The weight loss application 112 may be configured to store and retrieve data from one or more information sources, such as the data store 152. In addition, while a single server 101a is depicted in FIG. 1, it should be understood that one or more servers 101n may be included.

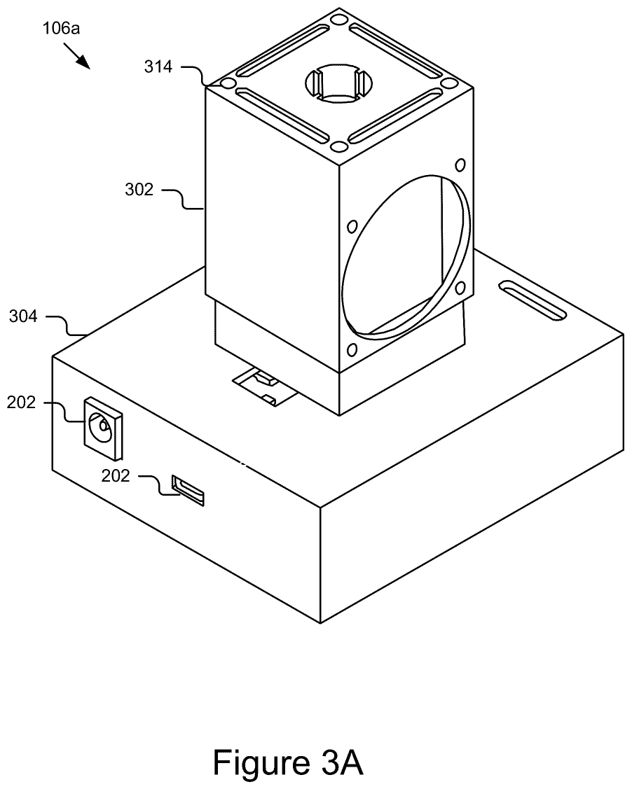

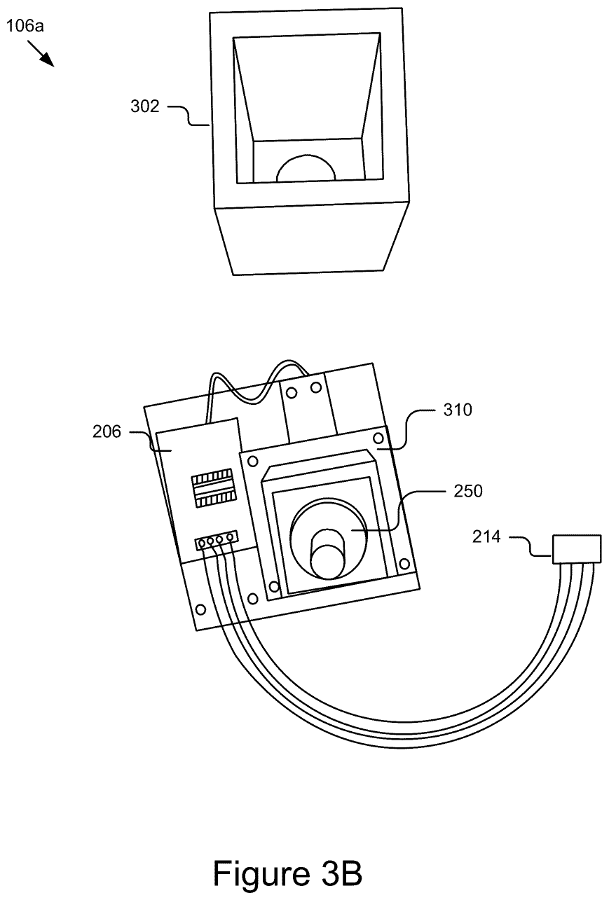

FIGS. 3A-3B are various views of one example of a weight loss device 106a. As shown in FIG. 3A, the weight loss device 106a includes a base 304 that houses the components of the weight loss device 106 (e.g., power supply 202, sensor 206, output devices 212, etc.) and a cover 302 that may protect and/or direct a scent emission of a scent solution 250 (not shown). In some implementations, as shown, the weight loss device 106a may be rectangularly shaped with the cover 302 configured to surround a vial or container of a scent solution 250. As shown in FIG. 3B, the base 304 may include a vial receiver 310 with a recess or place where a scent solution 250 can be positioned in order to accurately measure weight loss during testing. In some implementations, the vial receiver 310 may include one or more pressure sensors 206 positioned in such a way that the pressure sensors 206 can accurately detect weight changes in a vial containing the scent solution 250. As shown in FIG. 3A, the cover 302 may include perforations 314 or other perforation designs through which air can be drawn in and/or out of the cover 302 to emit scents similar to how a scent dispenser 102 dispenses scent.

As shown in FIG. 3B, in some implementations the weight loss device 106a may connect using a communication device 214 to one or more other components of the system 100. In some implementations, these connections can be hardwired as shown or wireless and connected to other devices of system 100 as described elsewhere herein. In some implementations, the weight loss device 106a may also include one or more other electronic interfaces, such as USB-C, USB, or other electronic port, which can be utilized to provide weight loss data or charge one or more batteries of the power supply 202, connect to other devices, plug the weight loss device 106 into a computer to update firmware, set settings, etc.

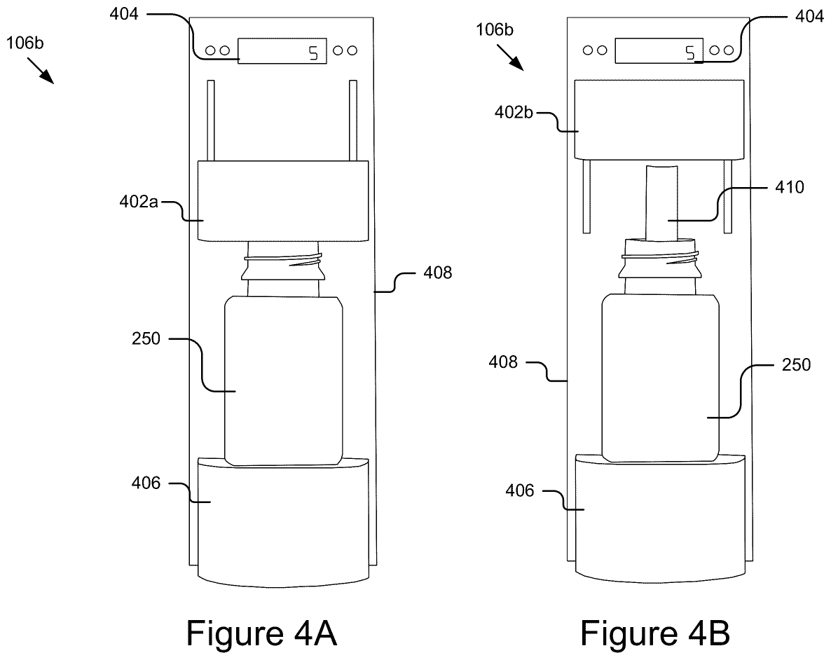

FIGS. 4A-4B are various views of another example of a weight loss device 106b. As shown in FIG. 4A, the weight loss device 106b may be in more of a vertical orientation as compared to the example weight loss device 106a described in FIGS. 3A-3B. As shown, the weight loss device 106b may include a body 408 that is positioned vertically, such as to allow for the weight loss device 106b to mount on a wall, although it should be understood that the weight loss device 106b could also be freestanding. The body 408 may have a solution base 406 protruding out of a bottom portion of the body 408. The solution base 406 may allow for a scent solution 250, such as a scent solution 250 stored with in a vial or container, to be positioned on a top surface of the solution base 406. The body 408 may further include a moveable scent top 402 that can slidable move up and down to engage with a portion of a vial or container of the scent solution 250. As shown in FIG. 4A, the moveable scent top 402 may be positioned in a closed position 402a where the moveable scent top 402 is moved downwards to encapsulate and retain a portion of the vial or container of the scent solution 250, such as a wick 410. As shown in FIG. 4B, the moveable scent top 402 may be positioned in an open position 402b where the moveable scent top 402 is moved upwards to expose and/or allow access to the vial or container of the scent solution 250. In the open position 402b, the moveable scent top 402 can be moved upwards to allow for easy removal or inspection of the scent solution 250, such as to switch out a scent solution 250 for testing. As shown in the examples, in some implementations, the weight loss device 106b may include visual data 404 that allows for easy identification of various data of the weight loss device 106b, such as a weight loss device identity, an indication of what type of scent is being tested, an indication of a current temperature setting, an indication of a current intensity setting etc. In some implementations, the visual data 404 may be displayed on an electronic display, such as an LCD screen or other electronic device acting as an output device 212 of the weight loss device 106. This allows for various information to be quickly viewed, set, and/or calibrated for a weight loss device 106 to begin testing. In some implementations, the weight loss device 106 may further include sensors 206 that can automatically determine an identity of the scent solution 250, such as an RFID reader that can read an RFID tag on a scent solution 250, or a camera or other imaging device that can automatically scan a unique label on the scent solution 250, etc.

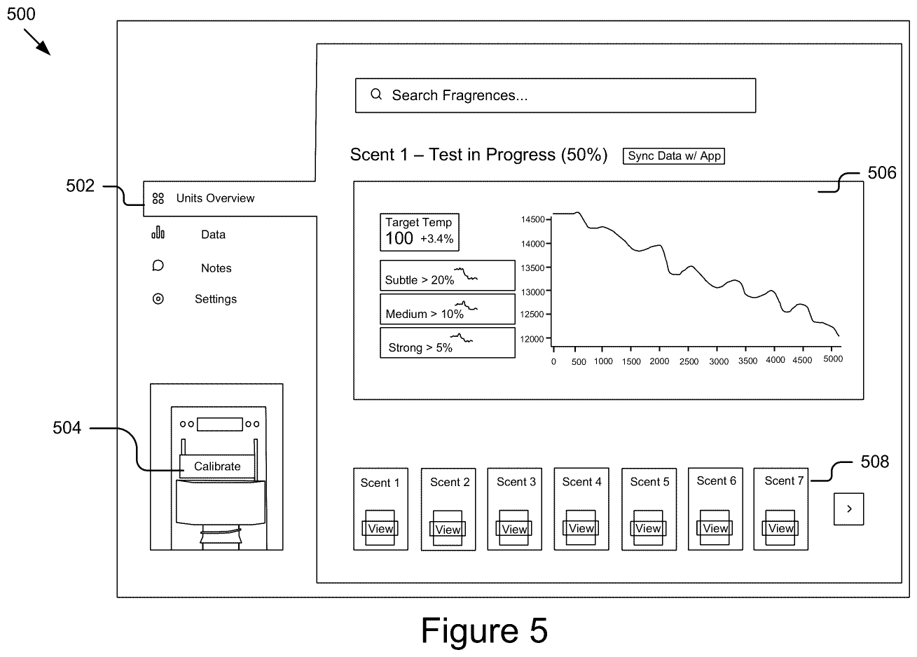

FIG. 5 illustrates an example interface 500 for viewing data from a weight loss device 106. As shown in FIG. 5, weight loss data can be captured by the weight loss device 106. In some implementations, the weight loss data may be shown as a weight loss of a scent solution 250 over time as shown by the weight loss data 506 in FIG. 5. The weight loss device 106 may be able to sample the various weight loss data in substantially real-time to capture real-time changes in the weight loss of the scent solution 250 as the scent is dispensed and create a visual, such as a graph of the data to communicate how that weight loss changes over time. This allows, among other things, the lifetime of the scent solution 250 in a container, such as a vial, to be determined at various scent dispenser settings. For example, in a testing environment, a weight loss device 106 can install a scent solution 250 at a first temperature setting and based on the usage data of the weight loss device 106 and the scent profile of the scent solution 250 based on previously gathered weight loss data, a lifetime of the scent solution 250 can be determined at a specific temperature. As the testing environment changes temperature settings, such as to increase or decrease a scent dispensation and gathers scent data at different settings for a scent solution 250, the lifetime of the scent solution 250 can be determined and change as the temperature settings change. A user 110 may be able to view the updated lifetime of the scent solution 250, such as in the user interface 500 on a computing device.

In some implementations, the weight loss application 112 may be able to identify additional trends based on analytics, such as at which temperatures and/or scent dispenser 102 settings, a scent solution 250 most efficiently disperses scent and/or extends scent lifetimes. Furthermore, different scent solutions 250 have different scent profiles and by using the weight loss device 106, unexpected weight loss data can be identified, such as that some scent solutions 250 have nonlinear lifetime changes when used at different scent dispensation settings. Using the weight loss device 106, these unexpected analytics can be identified for different scent solutions 250 in substantially real-time and can be provided to the users 110.

As shown in FIG. 5, the user interface 500 may allow a user 110 to view data of various different scent solutions 250 as shown by the scent selection option 508, where various different scent solutions can be selected to view different scent solutions 250 weight loss changes over time and/or at different temperature settings. In some implementations, the user interface 500 may be presented by the weight loss application 112 and may allow a user to interact with and/or change weight loss testing setups with one or more weight loss devices 106 during testing. In some implementations, a device calibration button 504 may be used to view specific data related to a weight loss device 106, such as testing parameters and/or calibration data of the specific weight loss device 106. The results of weight loss testing can be viewed in the menu 502 which may include unit overview showing the weight loss data at different temperatures, intensity (such as subtle, medium, strong), etc., data such as the raw collected data from the various sensors 206 of the weight loss device 106 over time and during testing, notes from various testers, and/or additional settings for a testing parameter.

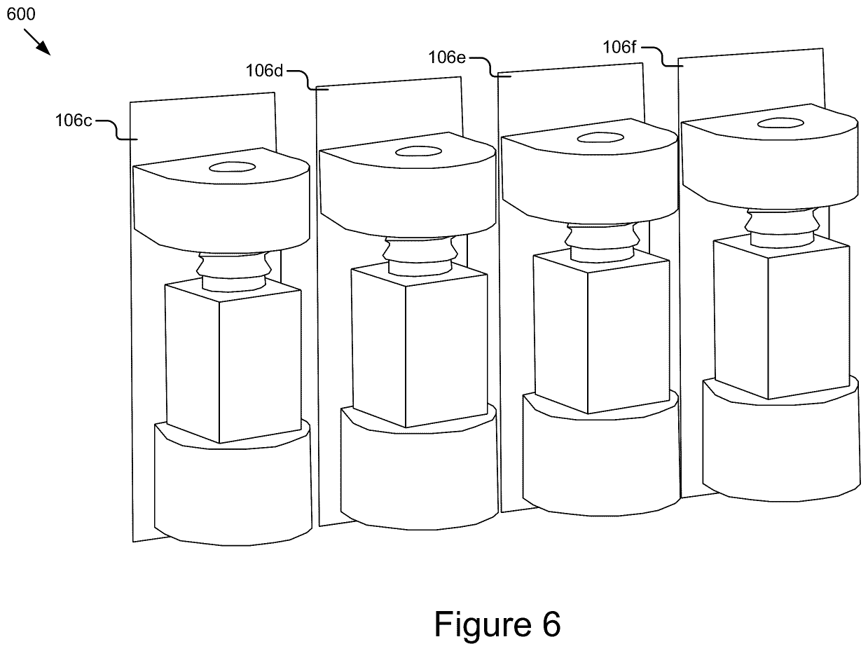

FIG. 6 illustrates an example array 600 of weight loss devices 106c-106g that can be testing different scent solutions. In some implementations, the weight loss application 112 may be able to view the gathered weight loss data from one or more weight loss device(s) 106 and may use machine learning to identify various traits, such as optimal temperature/intensity combinations for different scent solutions to maximize scent lifetime or scent detectability for different scent solutions 250, etc. Using the machine learning functionality, the weight loss application 112 can provide various insights based on a large volume of data gathered from an array of weight loss devices 106 testing different scent solutions 250 at different settings. It should be understood that various producers, manufacturers, developers, etc. could create an array of weight loss devices 106 to test different scent solutions 250. For example, as new scent solutions 250 are developed, they could be tested using an array of weight loss devices 106 to determine how scent solutions 250 change at different times, temperatures, intensities, etc.

As shown in FIG. 6, scent solutions 250 may be placed into the weight loss devices 106 and then data may be measured over time. The weight loss devices 106 may then be individually connected up to the weight loss application 112 and the weight loss devices 106 may begin gathering weight loss data of the scent solutions 250. These scent solutions may be the same scent solutions 250 and/or different scent solutions 250 and the weight loss devices 106 can be operating under the same testing settings, such as temperature, intensity, etc., or each weight loss device 106 can have different testing settings and operate independently of the other weight loss devices 106. The weight loss data may be presented, such as in the user interface 500 shown in FIG. 5 by the weight loss application 112 for determining various scent profiles of the various scent solutions 250. As shown in FIG. 6, multiple weight loss devices 106c-106f may be connected up to a weight loss application 112. It should be understood that any number of weight loss devices 106 may be connected to the weight loss application 112 and various scent profiles can be determined in substantially real-time as weight loss data is collected.

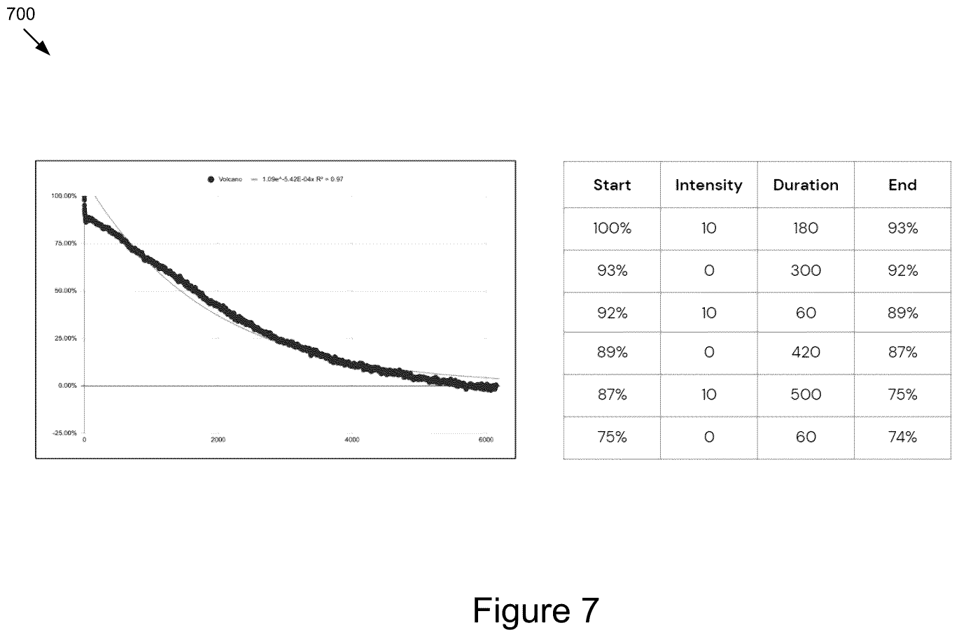

FIG. 7 illustrates another example system for weight loss testing. As shown in FIG. 7, the weight loss data can include start weight for measurements, intensity settings for the weight loss device 106, duration of the scent dispensation, and/or end weight for measurements. This weight loss data can be plotted to show an average scent solution 250 lifetime. As shown, in some implementations, the decrease in weight of the scent solution 250 is not a linear line over time and at different points, the weight loss and time are not linearly related. By capturing the weight loss data, the weight loss application 122 can identify the most efficient duration, intensities, or scent dispensation settings to prolong a scent solution 250 lifetime and/or achieve more efficient scent dispensing.

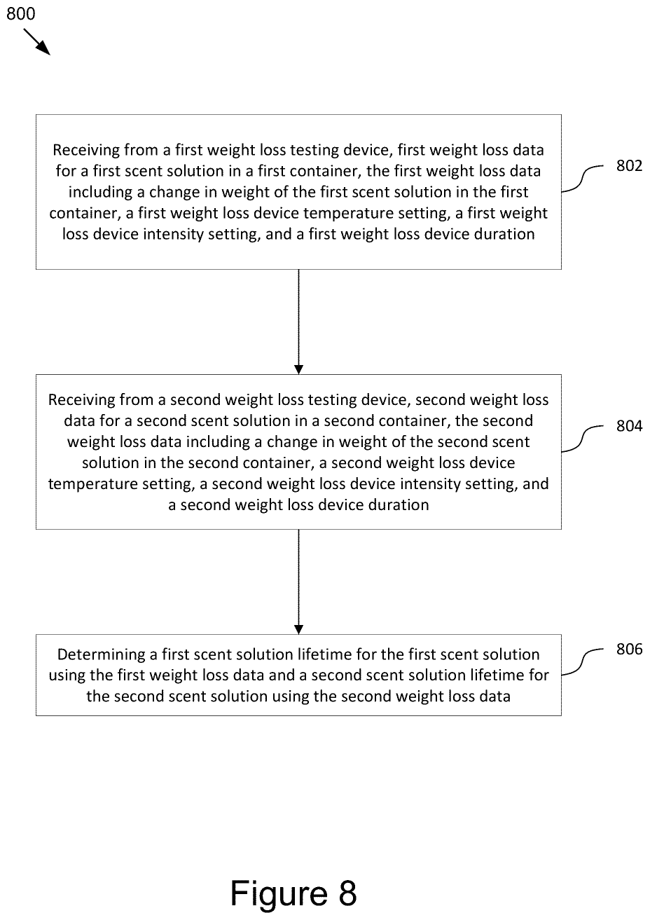

FIG. 8 illustrates a flowchart 800 of a method of determining a scent solution lifetime. As shown in FIG. 8, at 802, a weight loss application 112 may receive from a first weight loss testing device 106a, first weight loss data for a first scent solution 250 in a first container, the first weight loss data including a change in weight of the first scent solution in the first container and one or more of a first weight loss device temperature setting, a first weight loss device intensity setting, and a first weight loss device duration. The weight loss application 112 may receive this weight loss data in substantially real-time from the first weight loss testing device 106a, as well as any other weight loss testing devices 106 that are concurrently testing weight loss data for scent solutions 250.

For example, at 804, a second weight loss device 106b may be testing weight loss of a scent solution 250, as shown and the weight loss application 112 may receiving from the second weight loss testing device 106b, second weight loss data for a second scent solution 250 in a second container, the second weight loss data including a change in weight of the second scent solution in the second container, as well as a second weight loss device temperature setting, a second weight loss device intensity setting, and a second weight loss device duration. In some implementations, the first weight loss device 106a and the second weight loss device 106b may be testing a same type of scent solution 250, such as at different settings, where the first weight loss device 106a may have a first temperature setting and a first intensity setting and the second weight loss device 106b may have a second temperature setting and a second intensity setting that are different. In this situation, the two different devices may capture different data showing how the same scent solution 250 may act differently at different temperatures and/or intensities which may help inform on an overall lifetime of the scent solution 250 at different settings.

At 806, the weight loss application 112 can determining a first scent solution lifetime for the first scent solution 250a using the first weight loss data and a second scent solution lifetime for the second scent solution 250b using the second weight loss data. The scent solution lifetimes may be generated expectations of how long and/or how well a scent solution 250 will perform at specific settings (e.g., temperature, intensity, etc.) and when it is expected that the scent solution will run out at those specific settings. As additional weight loss data is gathered, that additional data can be added to a weight loss profile for a scent solution 250 and the weight loss application 112 can begin inferring, such as averaging out different collected data to determine overall scent lifetimes based on a larger sample size of collected scent data. In some implementations, the weight loss application 112 can infer various data about a scent solution 250 based on weight loss data, such as optimal settings to extend a scent solution 250 lifetime. For example, a user 110 or dispensing device 102 can receive scent setting recommendations to extend a scent solution 250 lifetime. In some implementations, a dispensing device 102 can automatically make scent setting changes based on a determined optimal setting for the scent solution 250 and/or various other user preferences.

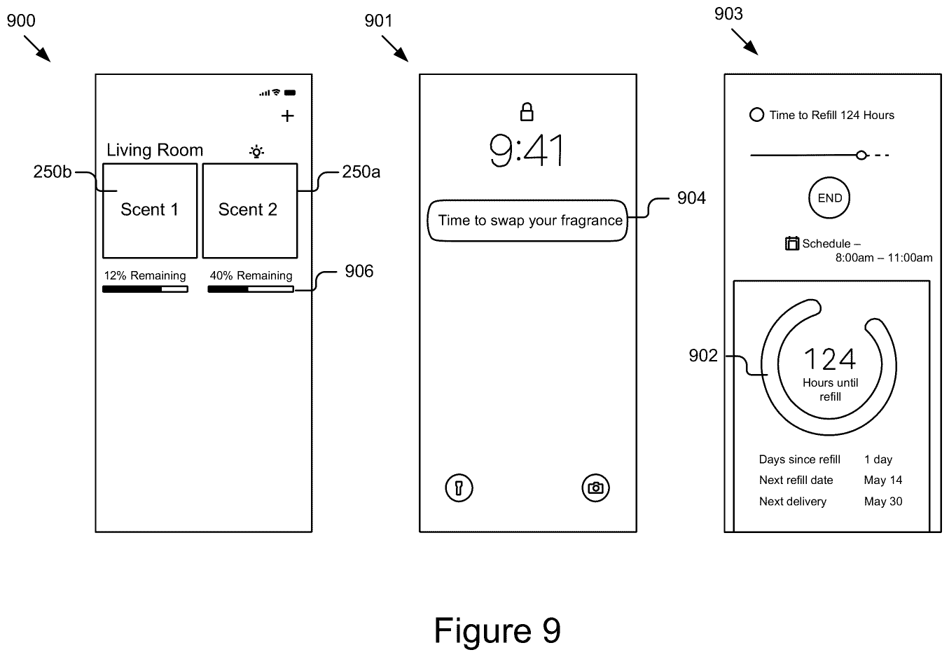

FIG. 9 illustrates example GUIs for scent dispensation. In some implementations, these GUIs may be presented on a display of a user device, such as mobile device, a computing device, etc. and a user can access these GUIs to control a scent dispenser 102. As shown, the GUI 903 may display an estimated lifetime 902 of a scent solution 250. In some implementations, the estimated lifetime 902 may be a duration of time left shown in hours/minutes. In further implementations as shown in GUI 900, various graphics 906 may be used to show percentages of scent solution 250 left. In some implementations, various recommendations for scent dispensation may be presented that provide more efficient lifetime or scent dispersion of a scent solution 250. In some implementations, a GUI 901 may be presented on a user device that provides a notification 904 to a user 110 when a scent solution 250 is running low. In some implementations, the weight loss application 112 may provide a push notification to a user device, while in further implementations, various automatic reordering routines may be used based on user preferences. As shown in the examples in FIG. 9, a user 110 may access a scent dispenser 102 to view various scent solutions 250a and 250b that are installed in the scent dispenser 102 as well as current scent levels (e.g., high, medium, low, etc.) to indicate to a user 110 how much scent solution 250 is left in each of fragrances of the scent dispenser 102.

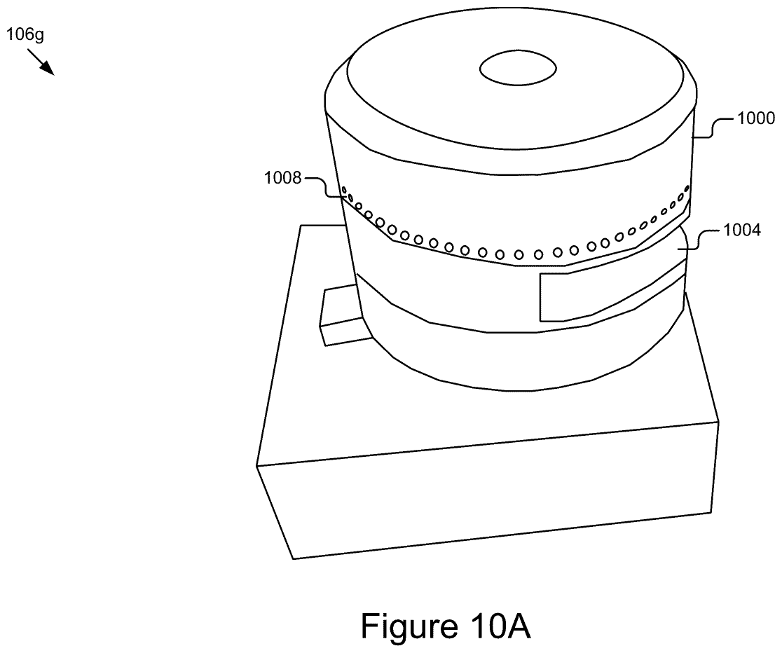

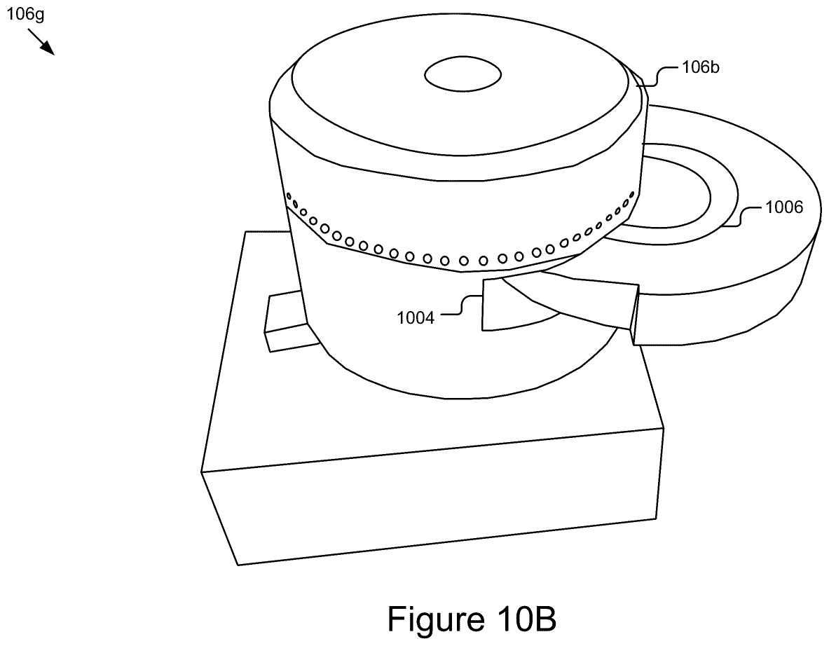

FIGS. 10A-10B are various views of another example of a weight loss device 106b. As shown in FIG. 10A, the weight loss device 106g includes a housing 1000 that houses the components of the weight loss device 106g (e.g., power supply 202, sensor 206, output devices 212, etc.). As shown in this example, the housing 1000 mayinclude a slot 1004 in which a scent solution 250 cartridge (not shown) including a scent solution 250 may be positioned. In some implementations, as shown, the weight loss device 106g may be cylindrically shaped and may be similar to a portable scent dispensing device, such as a vehicle scent dispenser, etc. In some implementations, the weight loss device 106g may include one or more pressure sensors 206 that can accurately detect weight changes in a scent cartridge containing the scent solution 250 in the form of an oil or gel. In some implementations, the weight loss device 106g may include one or more fans that create airflow over a scent solution cartridge (not shown) in order to disperse a scent solution, such as a gel or oil. The weight loss device 106g can detect changes in the weight loss of the scent solution 250 at different fan settings creating different airflows over the scent solution cartridge (not shown). As shown in FIG. 10A, the housing 1000 may include one or more perforations 1008 through which air can be drawn in and/or out of the housing 1000 to emit scents similar to how a scent dispenser 102 dispenses scent. As shown in FIG. 10B, a scent solution cartridge 1006 containing a scent solution 250 may be inserted into the slot 1004 for weight loss testing. This allows for both liquid scent solutions 250 to be used for weight loss testing, such as by using weight loss device 106a shown in FIGS. 3A-3B, FIGS. 4A-4B and scent cartridges 1006 with an oil or gel used for weight loss testing, such as by using weight loss device 106g shown in FIGS. 10A-10B.

As shown in FIGS. 10A-10B, in some implementations the weight loss device 106g may connect using the output device 212 to one or more other components of the system 100. In some implementations, these connections can be hardwired or wireless. In some implementations, the weight loss device 106g may also include one or more other electronic interfaces, such as USB-C, USB, or other electronic port, which can be utilized to provide weight loss data or charge one or more batteries of the power supply 202, connect to other devices, plug the weight loss device 106 into a computer to update firmware, set settings, etc.

In some implementations, where the user 110 is a producer/manufacturer, such as a fragrance house, etc. can use an array of weight loss devices 106 to infer various hedonics, such as how various scent solutions will smell in a room based on various scent settings. For example, using an array of weight loss devices 106 testing different scent settings for particular scent solutions 250, the user 110 can determine when particular scent notes are more prominent and/or begin to fall-off at different scent settings from testing. Furthermore, using machine learning and/or based on the known ingredients in different scents, the weight loss application 112 can learn how different scent solutions 250 are losing weight and direct developers on which ingredients common in different scents could help to change various aspects of the scent lifetime or scent experience. Additionally, the weight loss application 112 can use the weight loss data along with various purchasing data/other consumer data to simulate and connect differences between different scent solutions 250 to infer which products may be more successful and/or areas to market specific scent solutions 250. In some implementations, the weight loss data can help provide a further understanding of scent performance for a scent solution 250 and how that changes over time. This understanding of scent performance can be used by scent dispenser 102 to automatically adjust various scent settings over time without user intervention to maintain set scent intensity levels as the scent solution 250 begins to run out. For example, if a particular scent 250 has been shown in the weight loss data to have a scenting falloff at around 30% remaining, the scent dispenser 102 can increase the intensity when the amount of remaining scent solution drops below 30% so that the user 110 does not notice a falloff in scent experience, without requiring the user to make any changes. In additional implementations, using the weight loss data and various other data collected during the scenting experience, the weight loss application 112 can determine which scents in a scent solution 250 are binding to specific olfactory receptors and can tie that information into the development of new scents to focus on specific targeted scents being detected. Additionally, in some implementations, the weight loss application 112 can determine if specific scents are falling-off or no longer noticeable at specific points in the scent solution 250 lifetime and can use that to further develop new scents to can fill in those fall-off points to maintain a stable scenting experience.

The foregoing description, for purpose of explanation, has been described with reference to various embodiments and examples. However, the illustrative discussions above are not intended to be exhaustive or to limit the invention to the precise forms disclosed. Many modifications and variations are possible in view of the above teachings. The various embodiments and examples were chosen and described in order to best explain the principles of the invention and its practical applications, to thereby enable others skilled in the art to utilize the innovative technology with various modifications as may be suited to the particular use contemplated. For instance, it should be understood that the technology described herein can be practiced without these specific details in some cases. Further, various systems, devices, and structures are shown in block diagram form in order to avoid obscuring the description. For instance, various implementations are described as having particular hardware, software, and user interfaces. However, the present disclosure applies to any type of computing device that can receive data and commands, and to any peripheral devices providing services.

In some instances, various implementations may be presented herein in terms of algorithms and symbolic representations of operations on data bits within a computer memory. An algorithm is here, and generally, conceived to be a self-consistent set of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout this disclosure, discussions utilizing terms such as “processing,” “computing,” “calculating,” “determining,” “displaying,” or the like, refer to the action and methods of a computer system that manipulates and transforms data represented as physical (electronic) quantities within the computer system's registers and memories into other data similarly represented as physical quantities within the computer system memories or registers or other such information storage, transmission or display devices.

A data processing system suitable for storing and/or executing program code, such as the computing system and/or devices discussed herein, may include at least one processor coupled directly or indirectly to memory elements through a system bus. The memory elements can include local memory employed during actual execution of the program code, bulk storage, and cache memories that provide temporary storage of at least some program code to reduce the number of times code must be retrieved from bulk storage during execution. Input or I/O devices can be coupled to the system either directly or through intervening I/O controllers. The data processing system may include an apparatus may be specially constructed for the required purposes, or it may comprise a general-purpose computer selectively activated or reconfigured by a computer program stored in the computer.

The foregoing description has been presented for the purposes of illustration and description. It is not intended to be exhaustive or to limit the specification to the precise form disclosed. Many modifications and variations are possible in light of the above teaching. It is intended that the scope of the disclosure be limited not by this detailed description, but rather by the claims of this application. As will be understood by those familiar with the art, the specification may be embodied in other specific forms without departing from the spirit or essential characteristics thereof. Likewise, the particular naming and division of the modules, routines, features, attributes, methodologies and other aspects may not be mandatory or significant, and the mechanisms that implement the specification or its features may have different names, divisions, and/or formats.

Furthermore, the modules, routines, features, attributes, methodologies and other aspects of the disclosure can be implemented as software, hardware, firmware, or any combination of the foregoing. The technology can also take the form of a computer program product accessible from a computer-usable or computer-readable medium providing program code for use by or in connection with a computer or any instruction execution system. Wherever a component, an example of which is a module or engine, of the specification is implemented as software, the component can be implemented as a standalone program, as part of a larger program, as a plurality of separate programs, as a statically or dynamically linked library, as a kernel loadable module, as firmware, as resident software, as microcode, as a device driver, and/or in every and any other way known now or in the future. Additionally, the disclosure is in no way limited to implementation in any specific programming language, or for any specific operating system or environment. Accordingly, the disclosure is intended to be illustrative, but not limiting, of the scope of the subject matter set forth in the following claims.

What is claimed is:

1. A scent solution weight loss device, comprising:
a housing configured to retain a scent solution;
a sensor configured to detect weight loss data of the scent solution as the scent solution is emitted; and
an output device configured to provide the weight loss data to a weight loss application, wherein the weight loss application is configured to determine a scent solution lifetime based on the weight loss data.

2. The scent solution weight loss device of claim 1, wherein the weight loss application is configured to generate a scent solution profile using the weight loss data.

3. The scent solution weight loss device of claim 2, wherein the scent solution profile shows a change in scent solution over a measured time.

4. The scent solution weight loss device of claim 3, wherein the sensor is further configured to measure a temperature of the scent solution.

5. The scent solution weight loss device of claim 4, wherein the scent solution profile further includes the measured temperature of the scent solution.

6. The scent solution weight loss device of claim 1, wherein the housing further includes a heating element configured to heat the scent solutions to a temperature.

7. The scent solution weight loss device of claim 1, wherein the weight loss data includes a detected temperature, a detected intensity, and a detected duration.

8. The scent solution weight loss device of claim 7, wherein the weight loss application determines the scent solution lifetime based on the detected temperature, the detected intensity, and the detected duration.

9. A method of determining a scent solution lifetime, the method comprising:
receiving from a first weight loss testing device, first weight loss data for a first scent solution in a first container, the first weight loss data including a change in weight of the first scent solution in the first container, a first weight loss device temperature setting, a first weight loss device intensity setting, and a first weight loss device duration;
receiving from a second weight loss testing device, second weight loss data for a second scent solution in a second container, the second weight loss data including a change in weight of the second scent solution in the second container, a second weight loss device temperature setting, a second weight loss device intensity setting, and a second weight loss device duration; and
determining a first scent solution lifetime for the first scent solution using the first weight loss data and a second scent solution lifetime for the second scent solution using the second weight loss data.

10. The method of claim 9, wherein the first scent solution and the second scent solution are a same scent type.

11. The method of claim 10, further comprising:
determining an overall scent solution lifetime based on the first scent solution lifetime and the second scent solution lifetime; and
providing the overall scent solution lifetime to a scent application for display.

12. The method of claim 11, wherein the first weight loss data is a first graph showing the change in the first scent solution in the first container over the first weight loss device duration and the second weight loss data is a second graph showing the change in the second scent solution in the second container over the second weight loss device duration.

13. The method of claim 11, further comprising:
determining an optimal scent solution lifetime based on the overall scent solution lifetime.

14. The method of claim 9, wherein the first weight loss device temperature setting is different from the second weight loss device temperature setting.

15. The method of claim 9, wherein the first weight loss device intensity setting is different from the second weight loss device intensity setting.

16. The method of claim 9, wherein the first weight loss device duration is different from the second weight loss device duration.

17. A scent solution weight loss system, comprising:
a weight loss device configured to receive a scent solution and measure weight loss data of the scent solution over time;
a weight loss application configured to receive the weight loss data and generate a scent solution profile based on the weight loss data; and
a scent dispenser configured to receive the scent solution profile and adjust one or more scent dispenser settings based on the scent solution profile.

18. The scent solution weight loss system of claim 17, wherein scent dispenser settings include a temperature setting and an intensity setting and the scent dispenser adjusts one or more of the temperature setting and the intensity setting based on the scent solution profile.

19. The scent solution weight loss system of claim 18, wherein the weight loss application determines a scent solution lifetime based on the weight loss data.

20. The scent solution weight loss system of claim 19, wherein the scent dispenser adjusts one or more of the temperature setting and the intensity setting based on the scent solution lifetime to extend an operational time of the scent solution.
